(19) United States
(12) Patent Application Publication
(10) Pub. No.: US 2018/0235783 A1
(43) Pub. Date: Aug. 23, 2018

(54) DEVICE FOR CONNECTING PROTHESIS COMPONENTS TO A PROTHESIS SHAFT

(71) Applicant: Bernhard Sacherer, Friesenheim (DE)

(72) Inventor: Bernhard Sacherer, Friesenheim (DE)

(21) Appl. No.: 15/556,879

(22) PCT Filed: Mar. 7, 2016
(86) PCT No.: PCT/EP2016/000399

§ 371 Date: Sep. 8, 2017

(30) Foreign Application Priority Data
Mar. 9, 2015 (DE) 10 2015 002 882.0

(51) Int. Cl. A61F 2/76; A61F 2/80; A61F 2/60

(52) U.S. Cl. CPC A61F 2/76; A61F 2/80; A61F 2002/7818; A61F 2002/7625; A61F 2002/805; A61F 2/60

(57) ABSTRACT

The invention relates to a device for connecting prosthesis components (10) to a prosthesis socket (1) to which a connecting element (6) is associated, which connecting element is clamped against a proximally disposed and rotatably mounted counterpart-plate (15) and to which the prosthesis components (10) can be connected at least indirectly. In such devices the problem arises that, for example in the proximal joint of a leg stump, because of a muscular imbalance due to adduction or abduction of the stump on one side, considerable deviations from the natural position of the limb occur, such that also the position of the tip of a stump, both in the sagittal plane and in the frontal plane can deviate from the perpendicular (9) which is relevant for the structural analysis of a prosthesis. In order to nevertheless be able to construct a prosthesis along said perpendicular, according to the invention the connecting element (6) has a coupling element (11) mounted on a support (12, 17, 18) which extends substantially parallel to the central axis (8) of the prosthesis socket (1) in a plane (42) and is rotatable about said central axis (8). The distance between the coupling element (11) and the central axis (8) is adjustable, wherein the coupling element (11) is rotatable and lockable with respect to the support (12, 17, 18) on a second axis (41) extending substantially normal to the central axis (8) of the prosthesis socket (1). The prosthesis component (10) is mounted on the coupling element (10) and is rotatable and lockable about a third axis (9′) normal to the second axis (42).

### BACKGROUND

The invention relates to a device for attaching a prosthesis component to a prosthesis socket on which an attachment element is present via which the prosthesis component can be coupled to the prosthesis socket, wherein the attachment element is connected to a coupling element via a support and the prosthesis component is mounted on the coupling element, wherein the support lies substantially in a plane extending parallel to the central axis of the prosthesis socket and is rotatable about this central axis, and wherein the distance between the coupling element and the central axis is adjustable.

A device of this type is known from DE 10 2012 013 481.

### SUMMARY

It is known in orthopedics that prostheses have a prosthesis socket which is adapted to a body part, e.g. to a thigh stump, wherein the prosthesis socket has a wall substantially enclosing the body part, and wherein this prosthesis socket is provided, in particular at its distal end, with an attachment element by which further prosthesis components are coupled to the prosthesis socket, for example below-knee prostheses with an artificial knee joint, etc.

The known attachment elements are often in the form of an adapter plate which is cast in rigidly at the distal end of the prosthesis socket, e.g. in the form of a frequently used four-hole attachment plate or in the form of an M36 thread receiver, etc.

The body part, e.g. the thigh stump of a leg, can be covered with a silicone stocking (liner). In order then to produce a firm connection to the prosthesis socket, sealing lips, for example, can be provided on the liner that is to be pulled on over the stump. It is thus possible to retain the stump in the prosthesis socket by a vacuum, for example. The pump for this vacuum is integrated in the prosthesis, e.g. also in the attached prosthesis components, or a corresponding valve is provided on the prosthesis socket.

Moreover, in other embodiments, such a liner can be provided with various connection elements in its area adjacent to the tip of the stump, for example with a pin or a pull-in band, which are inserted into a holding device called a lock at the distal end of the prosthesis socket and are locked in place there. This holding device is usually cast into the prosthesis socket or is screwed distally onto the adapter plate.

In order that a stump, on which a liner provided with such a connection element lies, is able to be removed again from the prosthesis socket, the mechanism with which the pin or the pull-in band of the liner is secured in the lock must have elements that the prosthesis user can actuate from the outside and that are freely accessible for this purpose.

A basic consideration of an orthopedics engineer, in particular when producing a leg prosthesis or below-knee prosthesis, is that the prosthesis components are principally and connected to each other on the plumb line extending from the hip joint.

The problem then arises that, due to a muscular imbalance caused by unilateral adduction or abduction of the stump or due to flexion contractions, there may be considerable deviations from the natural position of the limb in the proximal joint of a leg stump, as a result of which the position of the tip of a stump may deviate from the plumb line both in the sagittal plane and also in the frontal plane.

For the desired static set-up of the prosthesis along the plumb line, prosthesis components attached at the distal end of a prosthesis socket in the area of the tip of the stump therefore have to be coupled to the attachment element via a connection that extends substantially horizontally.

Since the deviation of the longitudinal axis of an average thigh stump from said plumb line can amount to as much as 30°, and since a compensation of 8-10 mm to the plumb line is necessary for every 1° of deviation of the tip of the stump from the plumb line at the height of a knee joint, it is therefore necessary to compensate for more than 250 mm in an individual case.

To determine the details needed for a well-fitted prosthesis, particularly also as regards the desired static set-up, test prostheses are now first of all produced which are intended to function sufficiently safely and for as long as possible, even when used on a daily basis by the user.

In such test prostheses in particular, the connection of a prosthesis socket to the attached components usually entails the use of so-called cross supports, which have an adjustment path of ca. 30 mm. In such test prostheses, plates with rows of holes are alternatively used in combination with a ball pyramid whose angle is adjustable.

The starting point in each case is a four-hole receiver that is rigidly cast into the distal end of the prosthesis socket.

From this basis, an orthopedic engineer usually proceeds such that the compensation in the sagittal plane and the compensation in the frontal plane are carried out separately from each other.

A disadvantage of this is the need for very large and heavy structures which are built up by combination of several individual parts, but which nevertheless have only slight stability and, ultimately, only limited adjustment possibilities. The combining of individual connection elements to form an overall structure means that, in the displacement necessary for the adjustment, the screw connections with which the individual parts are connected often collide, and they also impede free access to the optionally present elements that are used to unlock the mechanism by which a pin or a pull-in band of a liner is held in a lock.

One possible way to avoid combined constructions of this kind in cases of serious mal-positioning is to position the attachment element on the prosthesis socket by a further add-on made of polyurethane foam. In this way, the starting position of an attachment element can be brought close to the desired position in the area of the plumb line relevant to the static set-up.

However, adhesive bonding of such a construction poses several risks, such as unknown chemical compatibility of the materials used. In later use of the prosthesis, there is also a considerable risk of its coming loose. To prevent this, corresponding constructions are often also wrapped in bandages, but this gives them a bulky shape.

In the initial management of a prosthesis user, it is sought in particular to alter the above-described mal-positioning of a leg stump by therapeutic measures. It is therefore also often necessary to regularly readapt the static set-up of a prosthesis. However, in the constructions in question, which are regarded as being unalterable because of their adhesive bonding, such readaptation is often dispensed with because the outlay for this is deemed to be too high.

The object of the invention is therefore to make available a connection of prosthesis socket to prosthesis component which, independently of the stump position, provides a readjustable and, if appropriate, also releasable defined connection of prosthesis socket and prosthesis component, in particular also for a test prosthesis.

According to the invention, this object is achieved by the features in the characterizing part of the accompanying main claim.

According to the invention, therefore, a device is provided for attaching a prosthesis component to a prosthesis socket on which an attachment element is present via which the prosthesis component can be coupled to the prosthesis socket, wherein the attachment element is connected to the coupling element via a support and the prosthesis component is mounted on the coupling element, wherein the support lies substantially in a plane extending parallel to the central axis of the prosthesis socket and is rotatable about this central axis, and wherein the distance between the coupling element and the central axis is adjustable. This device is characterized in that the coupling element is tiltable and lockable with respect to the support on a second axis extending substantially normal to the plane in which the support lies, and in that the prosthesis component mounted on the coupling element is rotatable and lockable about a third axis normal to the second axis.

An advantage of the invention is that a device according to the invention ensures that mal-positioning of the stump can be compensated via the adjustability of the coupling element with respect to the support, and that the coupling element can thus be arranged directly on a desired and ideal static line.

Whereas, in the previously known solutions, corresponding adjustments were made separately in the sagittal plane and in the frontal plane, as has been mentioned, it is now possible to perform a corresponding adjustment of the support with respect to a prosthesis socket simultaneously both in the circumferential direction and also in the radial direction.

Moreover, with such a design, it is also possible that the entire third axis runs substantially vertically, irrespective of whether the central axis of the prosthesis socket runs substantially vertically or deviates from such a vertical position.

The underlying concept of the invention is that the deviation of the position of the distal end of the stump from the abovementioned vertical line should be compensated on the shortest possible path. Under this aspect, it is relevant that the support is in principle rotatable about the central axis of the prosthesis socket, in order thereafter to be able to be fixed in any desired position.

The construction is so universal that it permits readjustment of the attachment device even when the mal-positioning is reduced, as is obtained in initial management by therapeutic measures. However, it is so mechanically stable that it is able to withstand stresses, even in the long term, and can thus also be used for a final prosthesis.

The variability of the present construction allows it to be used widely, and also independently of the amputation level of a stump. This permits higher piece numbers in production and thus ultimately also affords the possibility of more favorable or more economical manufacture.

In a particularly preferred embodiment, prosthesis function parts such as locks, valves or pull-in elements are mounted in the attachment element, in particular centrally, and are entrained in the rotation of the attachment element or of the support secured thereon. The access to the elements by which the securing mechanism or the pull-in device is actuated is thus ensured in every rotation position.

It is provided in particular that the support is configured as a planar plate element. This permits its relatively simple production, while at the same time ensuring its stability.

The support can also have a substantially U-shaped structure and can thus be secured at both sides on an attachment element.

However, it is also within the scope of the invention that the support is formed of two plate components which are secured laterally on the attachment element and which extend flat and substantially parallel to the central axis of the prosthesis socket. This leads to a high degree of stability of the construction and makes it possible to secure the coupling element between these plates. However, since the support is also secured laterally on the attachment element, it is also ensured that the attachment element can be designed in the hitherto customary manner and can receive locks, valves, pull-in elements, etc., in its area lying to the inside in the radial direction.

Particularly for quite considerable deviations of the distal tip of the prosthesis stump from the desired plumb line, the support and the plate parts from which it is composed are designed with a contour extending at least in part along the outer wall of the prosthesis socket. For this purpose, it is provided that there is not only a distal portion of the support extending normal to the central axis of the prosthesis socket and connected to the attachment element as described, but also that the support has a further portion which extends substantially along the outer surface of the prosthesis socket and which, at a proximal end of the support, comes to bear on the prosthesis socket and is connected there to the latter, which provides a further stabilizing and bearing action for the support.

The subject matter of this development of the invention has the advantage that the support is supported not only at the distal end of the prosthesis socket but also in the more proximal area, resulting in an improved force absorption overall, even in the case of a test prosthesis.

The forces can in particular be routed close to the plumb line and introduced into the prosthesis socket.

The embodiment of the invention as described above, with the support designed flat with plate elements, opens up almost unlimited adjustment possibilities in the sense of the displacement of the coupling element with respect to the prosthesis socket not only in the radial direction but in particular also in the axial direction, in order thereby to ensure the required bridging that has been discussed above.

The coupling element is preferably secured on the support, or on the plate elements forming the latter, by way of blind holes or through-holes. This results in a second axis which runs through these holes, both normal to the central axis of the prosthesis socket and also normal to a plane which extends parallel to the central axis and in which the support lies, about which second axis the coupling element is pivotable, such that the inclination of the coupling element with respect to the prosthesis socket, or to the central axis of the latter, can be modified. In the case of a substantially vertical position of the central axis, said second axis thus lies substantially horizontally.

The coupling element defines a third axis, which in turn lies normal to the second axis and about which the prosthesis components are mounted rotatably on the coupling element. This third axis is intended to extend substantially parallel to the plumb line during the use of a prosthesis. Thus, a prosthesis component can then be brought back to a position oriented in the sagittal or frontal plane.

Since all of the axes can each be fixed and locked, any desired position of the prosthesis socket relative to the axis of the prosthesis component can be compensated, the latter being rotated such that a movement plane customarily provided for such a component can be oriented on the frontal or sagittal plane of a user.

An essential aspect of the invention is the understanding that, in the previously known solutions, corresponding corrections were each carried out separately in the sagittal plane on the one hand and in the frontal plane on the other hand. By contrast, provision is now made to effect a corresponding orientation of the attachment element with respect to a prosthesis socket more or less directly in the direction of a drop line, i.e. the deviation of the distal end of the stump from the ideal plumb line can be bridged by the shortest path possible.

By virtue of the possibility of rotation of the attachment element or of the support, a direct positioning of the coupling element with respect to the central axis of the prosthesis socket in the direction of the drop line is possible and the coupling element remains in the axis lying normal to the central axis of the prosthesis socket. Proceeding from this, only the inclination of the socket has to be compensated, i.e. the third axis which in turn lies orthogonal to the second axis and in principle in the same plane as the first axis.

It is basically required that a coupling element during use lies substantially in a horizontal plane. This is also expedient, for example, for the attachment of knee joints, since a horizontal plane meets the conditions that form the basis of the design of the knee axis of industrially manufactured prosthetic knee joints. By rotation of the prosthetic component, this knee axis is then rotated about the third axis such that it lies in or parallel to the frontal plane and thus permits a movement of the below-knee prosthesis, which joins the prosthetic knee, in the sagittal plane, as is desired in order to obtain a natural gait.

In an alternative embodiment of the invention, the support is not designed as a plate but instead as a profile which, in accordance with the same principle as has been described above, extends along the surface of the prosthesis socket and bears proximally on the socket and is rotatable about the central axis of the prosthesis socket. A displaceable and in turn tiltable coupling element then sits on this profile.

In a particularly preferred embodiment, the coupling element is formed of two pivotable clamping devices which are spaced apart from each other. This affords the possibility that the aluminum tube conventionally used for the attachment of prosthesis components can be fitted directly onto the coupling element.

In an alternative embodiment with a reduced overall height, valuable structural height can be obtained with a very flat coupling element, for example in the case of knee exarticulation.

Special variants of these coupling elements have an eccentric setting for the inclination with respect to the support. An eccentric setting of this kind can be combined with an oblong hole, in particular on the support.

Furthermore, it is possible to provide a coupling element with a socket, e.g. for a conventional attachment pyramid.

### BRIEF DESCRIPTION OF THE DRAWINGS

Further advantages and features of the invention will become clear from the following description of illustrative embodiments. In the drawings:

FIG. 1 shows the side view of a device with a plate-like support,

FIG. 2 shows the rear view of a device according to FIG. 1,

FIG. 3 shows the side view of an embodiment with an alternative plate-like support,

FIG. 4 shows the side view of a device with a profile-like support,

FIG. 5 shows the schematic representation of a coupling element with two clamping devices at a distance from each other, FIG. 5A is a top view of the clamping device, and

FIG. 6 shows the side view of a coupling element with two axles for a low overall height.

### DETAILED DESCRIPTION OF THE PREFERRED EMBODIMENTS

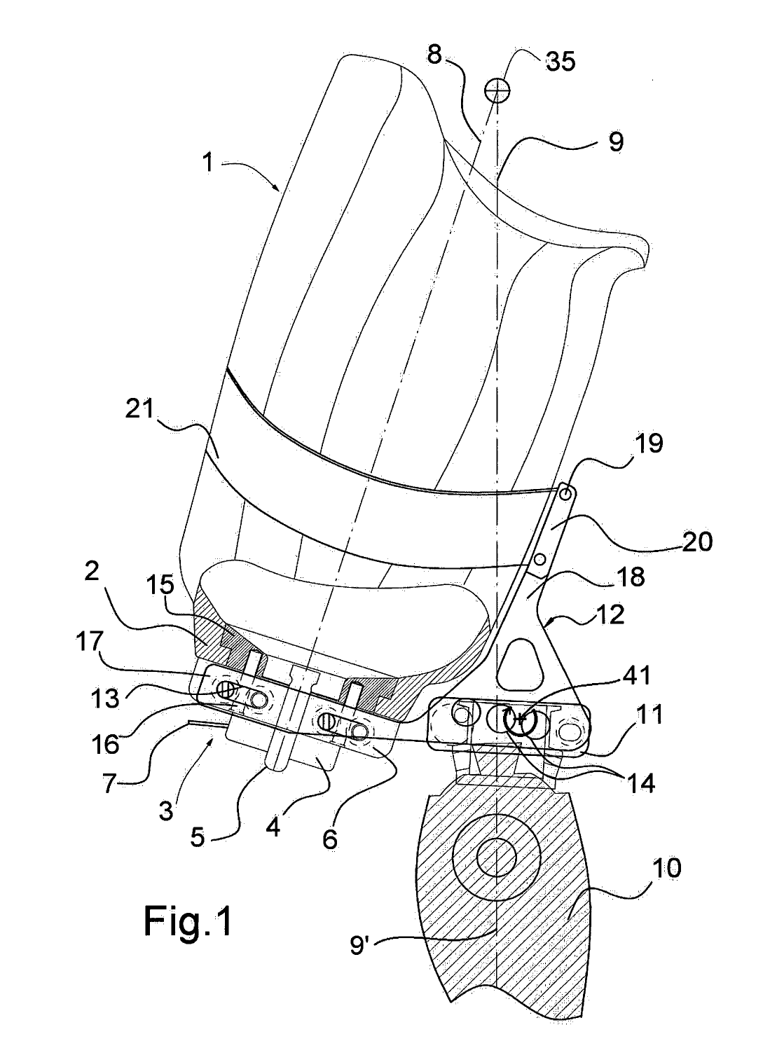

A prosthesis socket with prosthesis components attached thereto is shown in FIG. 1 in a schematic side view.

A prosthesis socket 1 can be seen which, in the example shown here, is adapted in terms of its shape to a thigh stump.

This prosthesis socket 1 has a wall 2 enclosing the thigh stump and, at its distal end 3, here the bottom end, is equipped with a holding device 4 as a prosthesis function part. This holding device 4 allows a user of the prosthesis to secure the prosthesis socket firmly on the associated body part, i.e. in the present case on a thigh stump. For this purpose, the thigh stump is covered with a liner in the form of a stocking which is made of silicone, for example, and which, at its distal end has a pin 5, for example, which is locked in the holding device 4 and thereby fixed.

The holding device 4 is mounted inside an attachment element 6, which is mounted rotatably at the distal end 3 of the prosthesis socket 1. The holding device 4 and the attachment element 6 are rotatable together.

In each rotation position, the holding device 4 can be actuated by the user of the prosthesis by an element 7, such that the connection of liner and holding device can be released.

It will be seen in FIG. 1 that the central axis 8 of the prosthesis socket 1 is inclined with respect to an ideal line 9 extending substantially vertically from the schematically symbolized hip joint 35. Thus, the tip of the prosthesis socket also does not lie on this ideal line. The deviation of the tip is induced by mal-positioning in the hip joint on account of the muscular adduction and/or abduction of the thigh stump, specifically in the sagittal plane and also in the frontal plane.

This ideal line 9 corresponds to the theoretical plumb line which is naturally present in a complete limb and which, in the case of a leg for example, runs through the hip joint, knee joint and ankle joint.

Since the distal end 3 of the prosthesis socket 1 does not lie on this ideal line 9, it is necessary to bridge the distance by which the distal end deviates from a position on said ideal line 9. The specific aim is to allow a prosthesis component 10 mounted on the distal end 3 of the prosthesis socket 1 to be arranged as close as possible to the ideal line 9.

In order to bridge the distance, the example shown in FIG. 1 is provided with a coupling element 11, by which the prosthesis component 10 is coupled to the prosthesis socket 1, wherein the coupling element 11 sits on a support 12.

The support 12 lies substantially in a plane in which the central axis 8 of the prosthesis socket also lies, or parallel to this plane. The support 12 thus extends substantially parallel to the central axis 8 of the prosthesis socket 1. In the example shown here, this plane lies in or parallel to the drawing plane.

In the example shown here, the support 12 is secured laterally on the attachment element 6 at the distal end 3 of the prosthesis socket 1, such that it is rotatable with the attachment element 6 about the central axis 8 relative to the prosthesis socket. The aim of this is to allow the ideal line 9 and the central axis 8 to lie in one plane. Short and, in particular, direct support paths are thus achieved.

In the example shown here, the attachment element 6 is secured on the prosthesis socket 1 or on the prosthesis socket wall 2 by the latter being clamped between the attachment element 6 and a counterpart 15, which bears internally on the prosthesis socket 1, via tensioning elements 16 in the form of screws. After the tensioning elements 16 have been loosened, the attachment element 6 can be rotated if necessary, in particular also together with the counterpart 15, and is then once again to be fixed to the wall 2 in a manner secure against rotation by the tensioning elements 16 being tightened.

For connecting the support 12 to the attachment element 6 in a rotationally fixed manner, the support 12 is secured laterally on the attachment element 6 via two oblong holes or offset holes 13.

The coupling element 11 is in turn secured on the support 12 via two of several through-holes or blind holes 14.

The distance of the coupling element 11 from the attachment element 6 and thus from the central axis 8 can be adjusted via the holes 13 or through the choice of a matching pair of though-holes or blind holes 14. The coupling element is thus moved substantially within the abovementioned plane, which here extends parallel to the drawing plane.

In addition, however, the angle position of the coupling element 11 can also be modified, such that the latter is adjustable about an axis 41. This axis 41 is normal to the plane defined by the support 12.

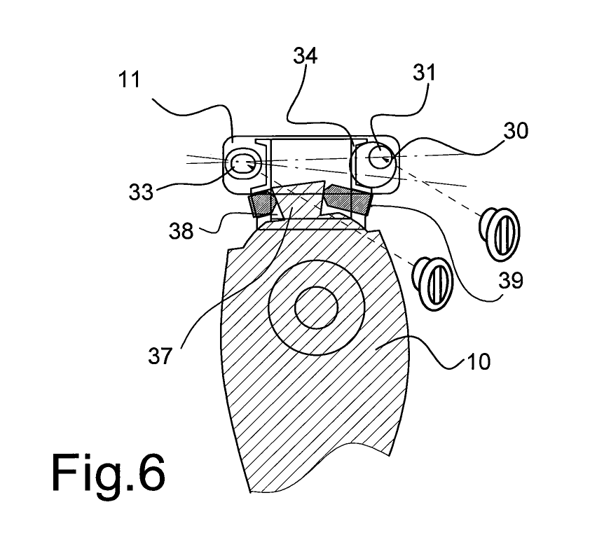

In order to secure the prosthesis component 10 to the coupling element 11, it has at its proximal end, as can be seen from FIG. 6, a conical polygon 37 that is inserted into a receiving bore 38 on the coupling element 11. Inside this receiving bore 38, the conical polygon 37 is then fixed by a plurality of substantially radially oriented clamping screws 39. The clamping screws 39, which are also designed in particular as set screws, have an inclination such that the prosthesis component 10 is pulled with its conical polygon 37 into the receiving bore and is thus connected firmly to the coupling element 11.

In the example shown in FIG. 1, the support 12 is designed as a plate-shaped and therefore substantially flat component. It has a portion 17 in the area of the distal end 3, which is connected to the attachment element 6, and a portion 18, which extends substantially along the wall 2 of the prosthesis socket 1 in the proximal direction thereof. At its proximal end, this portion 18 is coupled by a screw union 19 with an element 20 onto a fixing band 21 extending around the prosthesis socket.

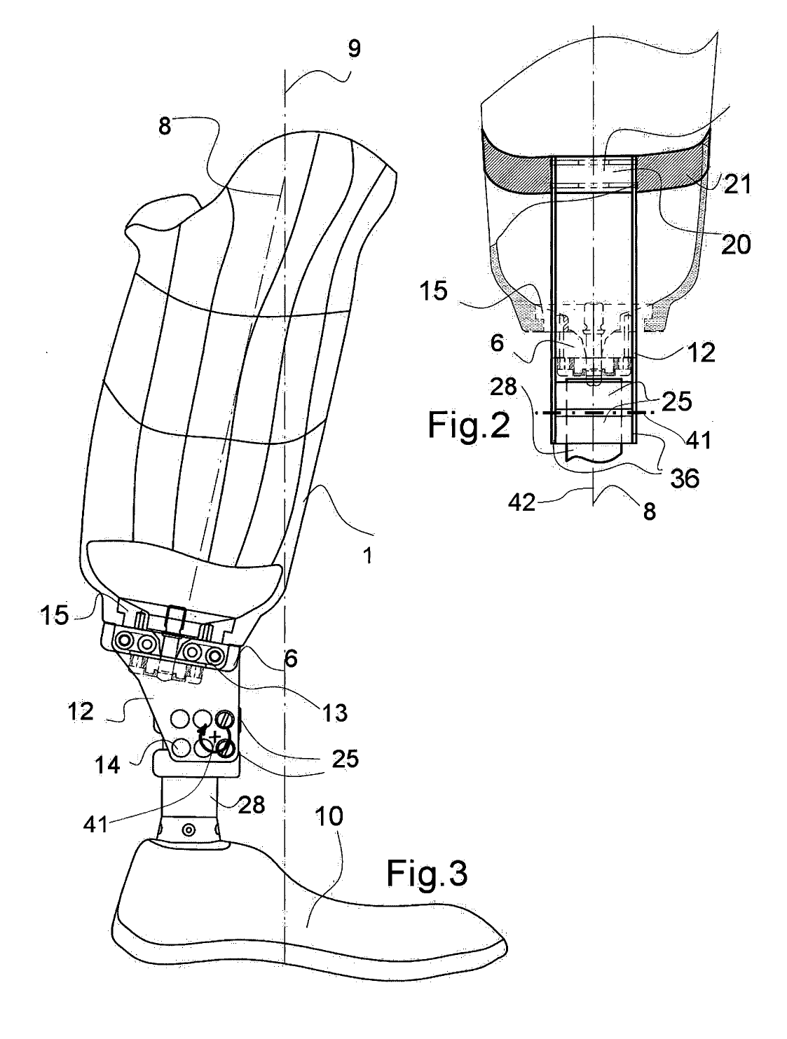

It will be seen from FIG. 2 that the element 20 is a tensioning element via which the fixing band 21 is drawn taut around the wall 2 of the prosthesis socket 1, such that it forms a securing element for the portion 18, which thus engages laterally on the wall 2 of the prosthesis socket 1.

It will also be seen in particular from FIG. 2 that the support 12 is formed of two parallel plates 36 between which there lie the attachment element 6 and the coupling element 11, the latter in this example having two clamping elements 25. A tube 28, whose function is explained in more detail below, is held and clamped by the clamping elements 25. The two clamping elements 25 lie parallel to the axis 41 about which the coupling element is pivotable.

In the area where the two portions 17 and 18 meet as shown in FIG. 1, the support 12 is provided with the multiplicity of holes 14 discussed above, which are used to receive the coupling element 11.

As the coupling element 11 is secured on the support 12 by way of these holes 14, it is pivotable about a second axis 41 (lying normal to the drawing plane) which here extends perpendicularly with respect to the central axis 8 of the prosthesis socket 1. The central axis 8 thus lies in the plane 42 in which, or parallel to which, the support 12 lies, and within which the coupling element 11 is therefore tiltable. In the coupling element 11 itself, a third axis is defined (which in turn lies in the drawing plane) about which the prosthesis component 10 can then be rotated. This third axis again extends perpendicularly with respect to said second axis and ideally with the ideal line 9. It is therefore designated by reference sign 9.

As has been mentioned, said holes 14 are arranged substantially like a grid, thus affording the possibility that the coupling element 11, via which a prosthesis component 10 is secured on the support 12, can be secured in this area at any desired location lying as close as possible to the ideal line 9 discussed above. In this way, the prosthesis component 10 attached to the prosthesis socket 1 can be arranged on the ideal line 9, and the user of such a prosthesis can thus achieve a pattern of movement that is as natural as possible.

As has been explained, it will be seen from FIG. 2 that the support 12 is formed of two plate-like components which extend parallel to each other and are of substantially identical form, and between which the attachment element 6 and also the coupling element 11 are then secured. The two components 36 of identical form, which thus form the support 12, are then arranged to both sides of the plane extending through the central axis 8 and are mounted to the left and right, respectively, onto the attachment element 6 at the distal end 3 of the prosthesis socket 1.

FIG. 3 shows an alternative embodiment of a support 12.

It will be seen here that the inclined setting of the prosthesis socket 1, and therefore the deviation of the prosthesis tip from the desired ideal line 9, is less than in the embodiment according to FIG. 1. In the support 12, therefore, it is possible to dispense with the portion 18 which extends along the outer wall of the prosthesis socket 1 and permits further support at the proximal end thereof. Therefore, the holes 14 with which the coupling element is connected via the clamping elements 25 to the support 12, and via the latter to the attachment element 6, can be arranged closer to said attachment element 6. However, in this case too, the distance of the coupling element 11 from the attachment element 6 can be modified both in the axial direction and also in the radial direction.

In the area in which the supports 12 are secured on the attachment element 6, the holes provided for this purpose are designed as oblong holes 13, such that the support 12 is also displaceable in its position relative to the attachment element 6, and, furthermore, the securing holes 14 for the coupling element are arranged in groups next to each other, such that, in this case too, the adjustability of the coupling element can be achieved through the choice of suitable holes 14.

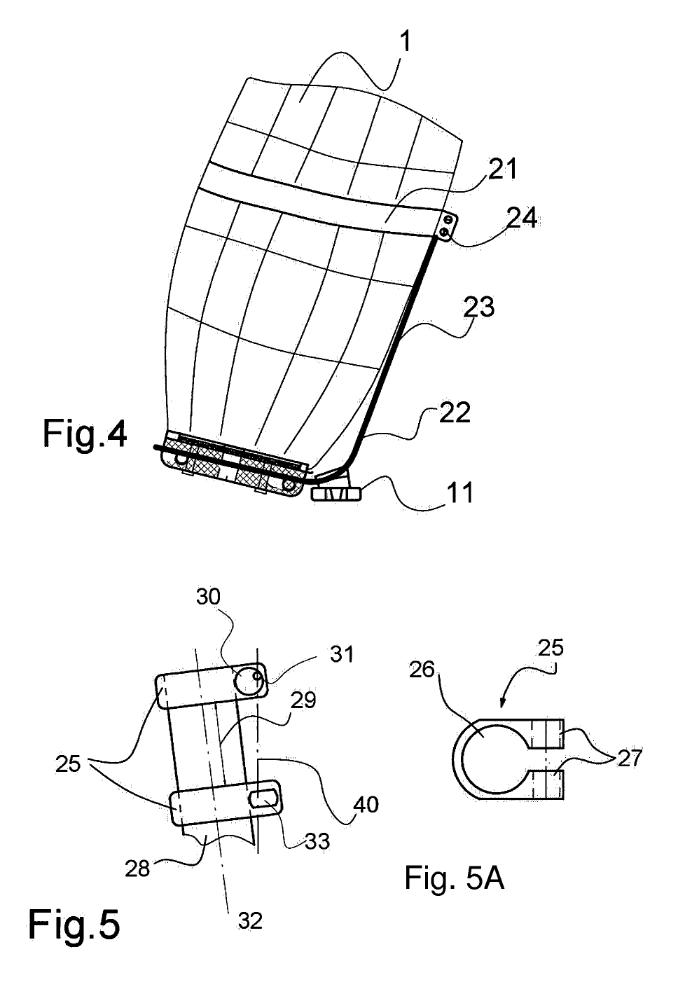

FIG. 4 shows a further alternative embodiment of a support. In this embodiment, the support 22 is formed in one piece from a curved sheet of metal or a curved profile bar, and the attachment element 11 sits on this support 22 and, if necessary, can be moved along the latter.

In this embodiment also, the support 22 is designed with a contour extending at least in part along the outer wall of the prosthesis socket, and the corresponding portion 23 is coupled to the wall 2 of the prosthesis socket 1 via a bracket 24 on a fixing strap 21 and is thereby connectable to the prosthesis socket 1.

FIG. 5 shows a special coupling element. The latter is formed of two clamping devices 25, as are shown in the plan view in FIG. 5A. Such a clamping device 25 has a through-opening 26, of which the circumference can be reduced by the pulling-together of two cheeks 27, and a tube 28 inserted in the through-opening 26 can thereby be securely clamped.

Prosthesis components are provided with such a tube in their attachment area, such that a prosthesis component can be secured to the coupling element 11 via this tube 28 and thereby secured on the attachment element 6 of the prosthesis socket.

The inclination of the tube 28 with respect to the support 12 can be determined by the choice of the matching through-holes or blind holes 14, as can be seen in a possible variant in FIG. 3.

As can be seen in FIG. 5, the clamping devices 25 are at a distance 29 from each other, such that the tube 28 is held at two points clearly spaced apart from each other. A corresponding stability can thus be achieved, which is important for the attachment of prosthesis components to the prosthesis socket.

Eccentric pieces 30, which can be rotated in the through-holes or blind holes 14 discussed above, are moreover inserted into the cheeks 27. By way of this rotation of the eccentric pieces 30, the axis 40 extending through securing pins 31 on the eccentric pieces 30 is changed in terms of its inclination with respect to the central axis 32 of the held tube 28, as a result of which, ultimately, the attachment angle for the tube 28 can be set with respect to the support 12.

Thus, the rough angle position of the central axis 32 or of the tube 28 is effected through the choice of the pair of holes 14 on the support 12 that receive the eccentrics 30, and the fine adjustment is then effected by rotation of the eccentrics 30.

In an alternative embodiment, order to avoid unwanted strain between two eccentric adjustments, an oblong hole 33 can be provided in the clamping device 25, with a combination of an eccentric 30 and of an oblong hole 33 also being possible.

The central axis of the tube 28 is the above-discussed third axis about which the tube 28, and thus the prosthesis component 10 supported by the latter, can be brought to any desired rotation position within the cheeks 27 and can then be fixed by the cheeks 27 being brought together.

FIG. 6 shows a variant of an inclinable coupling element 11 which has a low overall height. It shows once again the combination of an oblong hole 33 and of an eccentric 30 in connection with a rotatably mounted pyramid receiver 34, wherein a modified distance, caused by rotation of the eccentrics, between the elements guided through these holes can be compensated by the oblong hole 33 when the distance between the receiving holes 14 on the support plates 12 is at the same time unchanged. The pyramid receiver 34 is thus likewise clamped inside the coupling element via two cheeks 27.

In the case of a prosthesis socket of the kind used for stumps with amputation near the joint, for example a knee exarticulation, this embodiment makes it possible to secure further prosthesis components with low overall height and in a stable and yet widely adjustable manner.

Since all of the devices described above are entirely without adhesive bonding, a prosthesis socket can remain directly on the stump for most adaptation work, as a result of which the adaptation of a prosthesis is considerably quicker and also more precise than with the previously known devices described in the introduction.

Therefore, with a device according to the invention, the position of the stump axis, and therefore the adapter position, can be very easily transferred with precision from a test prosthesis to the positive mold for a final prosthesis which is to be produced thereafter, this adapter allowing the static set-up to be transferred very precisely to the final prosthesis without additional work.

Whereas hitherto, in the production of a final prosthesis with an adhesively bonded attachment, it has been necessary for the latter to be covered with a second molding, which causes additional weight and makes subsequent readjustment impossible, the use of a device according to the invention does away with this work. Moreover, in a prosthesis according to the invention, the mal-positioning of a stump, which changes over the course of use of this prosthesis, can be readjusted at any time.

### LIST OF REFERENCE SIGNS

1 prosthesis socket
2 wall
3 distal end
4 holding device
5 connection element liner—lock
6 attachment element
7 operating element for lock
8 central axis
9 ideal line
9′ third axis
10 prosthesis component
11 coupling element
12 support
13 oblong holes/row of holes
14 holes on the support 12
15 inner counterpart plate
16 tensioning element
17 distal portion
18 proximal portion
19 screw union
20 tensioning element
21 fixing band
22 support
23 proximal portion
24 bracket
25 clamping device
26 through-opening
27 cheek
28 tube
29 distance
30 eccentric pieces
31 axle/securing pins
32 central axis
33 oblong hole
34 pyramid receiver
35 hip joint
36 plates
37 conical polygon
38 receiving bore
39 clamping screws
40 axis
41 second axis
42 plane

1. A device for attaching a prosthesis component (10) to a prosthesis socket (1), comprising an attachment element (6) on the prosthesis socket (1) via which the prosthesis component (10) is couplable to the prosthesis socket, wherein the attachment element (6) is connected to a coupling element (11) via a support (12, 17, 18) and the prosthesis component (10) is mounted on the coupling element (11), the support (12, 17, 18) lies substantially in a plane (42) extending parallel to a central axis (8) of the prosthesis socket (1) and is rotatable about said central axis (8), and a distance between the coupling element (11) and the central axis (8) is adjustable, the coupling element (11) is tiltable and lockable with respect to the support (12, 17, 18) on a second axis (41) extending substantially normal to the plane (42) in which the support (12, 17, 18) lies, and the prosthesis component (10) mounted on the coupling element (11) is rotatable and lockable about a third axis (9′) extending normal to the second axis.

2. The device as claimed in claim 1, wherein the attachment element (6) is mounted on the prosthesis socket (1) so as to be rotatable and lockable with respect to the central axis (8).

3. The device as claimed in claim 2, wherein prosthesis functional parts (4) including at least one of locks, valves or pull-in elements are mounted in the attachment element (6) and are entrained with a rotation of the attachment element (6).

4. The device as claimed in claim 1, wherein the support is configured as a flat plate element (12).

5. The device as claimed in claim 1, wherein the support is configured as a profile (22).

6. The device as claimed in claim 1, wherein the support is formed of two plate components (36) which are secured laterally on the attachment element (6) and which extend flat and are substantially parallel to the central axis (8) of the prosthesis socket (1).

7. The device as claimed in claim 1, wherein the support (12) has a portion (18) which extends substantially along an outer surface of the prosthesis socket (1) and which, at a proximal end, comes to bear on the prosthesis socket.

8. The device as claimed in claim 1, wherein blind holes or through-holes (14) are provided on the support (12) to secure the coupling element (11).

9. The device as claimed in claim 1, wherein the coupling element (11) is formed of two pivotable clamping devices (25) which are spaced apart from each other and receive a tube (28) of a prosthesis component.

10. The device as claimed in claim 1, wherein the coupling element (11) is formed of a plate with two mutually pivotable connectors (30, 33) and is equipped with a pyramid receiver (34).
